(19) United States
(12) Patent Application Publication
(10) Pub. No.: US 2005/0025806 A1
(43) Pub. Date: Feb. 3, 2005

(54) Sustained release pharmaceutical composition

(76) Inventors: Malcolm Brandon, Bulleen (AU); Serge R. Martinod, Groton, CT (US)

Correspondence Address: MORGAN LEWIS & BOCKIUS LLP, 1111 PENNSYLVANIA AVENUE NW, WASHINGTON, DC, 20004, US

(21) Appl. No.: 10/895,957

(22) Filed: Jul. 22, 2004

Related U.S. Application Data
(63) Continuation-in-part of application No. PCT/AU03/00071, filed on Jan. 23, 2003.
(60) Provisional application No. 60/351,440, filed on Jan. 24, 2002.

(51) Int. Cl. A61K 48/00; A61K 38/22; A61K 38/19; A61K 31/56

(52) U.S. Cl. 424/423; 514/44; 514/12; 514/169; 424/85.1

(57) ABSTRACT

A sustained release delivery apparatus including a silicone support material formed from a methyl-vinyl siloxane polymer including a fumed silica as a reinforcing filler; a pharmaceutically active composition carried in or on the silicone support material; the pharmaceutically active composition including at least one growth and/or reproduction-associated pharmaceutical component; analogue thereof or derivative thereof; and a carrier therefor.

### CROSS REFERENCE TO RELATED APPLICATIONS

This application is a Continuation-in-Part of PCT/AU03/00071, filed Jan. 23, 2003, which claims priority to U.S. Provisional Application No. 60/351,440, filed Jan. 24, 2002, both of which are incorporated by reference herein in their entirety.

### DETAILED DESCRIPTION

The present invention relates to sustained release pharmaceutical compositions, to a method for the preparation thereof and to use thereof inter alia in improving growth characteristics in animals including humans. More specifically, the present invention relates to a sustained release pharmaceutical composition, which a growth-related pharmaceutical active.

A number of drug delivery systems are known in the prior art.

For example, a controlled drug-release preparation using as a carrier a hydrophobic polymer material, which is non-degradable after administration into the living body. There are two methods of controlling release of a drug from such preparation; one, using an additive such as an albumin, and another, by forming an outer layer consisting of hydrophobic polymer alone.

However, where a disease indication requires the achievement of a high threshold blood plasma level and/or requires the delivery of multiple pharmaceuticals and/or requires sustained release to be continued over an extended period at high levels, the drug delivery systems known in the prior art generally exhibit insufficient drug carrying capacity.

In addition, techniques known in the prior art for producing sustained release implants utilise a silicone based technology based on an extrusion system.

Difficulties have been encountered in attempting to scale up such techniques to commercial volumes. Difficulties have also been encountered in applying such extrusion techniques to pharmaceutical actives such as Recombinant Porcine Somatotropin (rPST). For example, such activities interfere with silicone chemistry due to their chemical composition or exhibit temperature sensitivity.

Further, sustained release drug delivery systems have been proposed for delivery of, for example, growth hormones. However, treatments providing a sustained or constant dosage of growth hormone, such as the Alza-type osmotic pump system, have been found to be deleterious to growth and leading to reduced food intake and other negative results in animals so treated.

This has lead to treatments via daily injections or injections every second day to provide a pulsed treatment. Such treatments are, however, recognised as sub-optimal and highly labour intensive.

It is, accordingly, an object of the present invention to overcome or at least alleviate one or more of the difficulties and deficiencies related to the prior art.

Accordingly, in a first aspect of the present invention, there is provided a sustained release delivery apparatus including
a silicone support material formed from a methyl-vinyl siloxane polymer including a fumed silica as a reinforcing filler;
a pharmaceutically active composition carried in or on the silicone support material;
the pharmaceutically active composition including at least one growth and/or reproduction-associated pharmaceutical component; analogue thereof or derivative thereof; and
a carrier therefor.

It has surprisingly been found that the sustained release delivery apparatus according to the present invention may be utilised to deliver pharmaceutical actives, for example growth hormones, which heretofore have proved ineffective and/or sub-optimal in a sustained release form.

The sustained release delivery apparatus may take the form of a coated molded or extruded rod or dispersed matrix structure. The sustained release delivery apparatus may be of the type described in International patent applications PCT/AU02/00865, PCT/AU02/00866 and PCT/AU02/00868 and Australian provisional patent application PR9515 and to Applicants, the entire disclosures of which are incorporated herein by reference.

A sustained release mini-implant or pellet is preferred. A plurality of mini-implants or pellets is particularly preferred.

Accordingly, in a preferred aspect of the present invention, the sustained release apparatus includes a plurality of sustained release mini-implants or pellets;
each mini-implant including
   a silicone support material formed from a methyl-vinyl siloxane polymer including a fumed silica as a reinforcing filler; and
   a pharmaceutically active composition carried in or on the silicone support material;
   the pharmaceutically active composition including
      at least one growth and/or reproduction-associated pharmaceutical; analogue thereof or derivative thereof; and
      a carrier therefor;
each implant being of insufficient size and/or payload individually to provide a predetermined desired threshold blood level of pharmaceutical active for treatment of a selected growth and/or reproduction-associated indication.

The number and/or size of the mini-implants or pellets may be selected to improve one or more of the characteristics described above.

Applicants have surprisingly found that such improvements may be achieved wherein the mini-implants provide, in use, less than the daily equivalent injectable dosage, for example wherein the mini-implants provide, in use, approximately half the daily equivalent injectable dosage. This may represent a significant saving in cost to the user.

In general, the sustained release apparatus may include approximately 5 to 20 mini-implants. Each mini-implant may have an axial length of approximately 1 to 40 mm, more preferably approximately 1 to 5 mm, most preferably approximately 2 mm.

The sustained release delivery apparatus according to the present invention preferably exhibits loading capacities of pharmaceutical active of 10% to 65% by weight, more preferably 15% to 50% by weight, most preferably approximately 20% to 40% by weight, based on the total weight of the pharmaceutically active composition.

Preferably the sustained release delivery apparatus may provide approximately zero order release of pharmaceutical active.

The pharmaceutically active composition, as described above, includes
at least one growth and/or reproduction-associated pharmaceutical component.

The pharmaceutical component may be selected from one or more of cytokines, hormones, hormones (eg. growth hormone, growth hormone releasing factor, calcitonin, leuteinizing hormone, leuteinizing hormone releasing hormone, and insulin), hormone agonists and antagonists (e.g. LHRH), growth factors (eg. somatomedin, nerve growth factor, insulin-like growth factor (IGF)), neurotrophic factors, fibroblast growth factor, and hepatocyte proliferation factor; growth factors, live vectors and live cells secreting growth hormones and RNA and DNA coding for growth hormones.

More preferably the pharmaceutical active includes one or more selected from the group consisting of cytokines, hematopoietic factors, hormones, growth factors, neurotrophic factors, fibroblast growth factor, and hepatocyte proliferation factor; and cell adhesion factors.

Recombinant porcine somatotropin (rPST) is particularly preferred.

The pharmaceutically active composition of the present invention may contain two or more drugs depending on the indication and mode of application.

The pharmaceutically active component may accordingly further include one or more actives selected from the group consisting of:

| | |
|---|---|
| Acetonemia preparations | Anabolic agents |
| Anaesthetics | Analgesics |
| Anti-acid | Anti-arthritic |
| Antibodies | Anti-convulsivants |
| Anti-fungals | Anti-histamine |
| Anti-infectives | Anti-inflammatories |
| Anti-microbials | Anti-parasitic |
| Anti-protozoals | Anti-ulcer |
| Antiviral pharmaceuticals | Behaviour modification drugs |
| Biologicals | Blood and blood substitutes |
| Bronchodilators and expectorants | Cancer therapy and related |
| Cardiovascular pharmaceuticals | Central nervous system pharma |
| Coccidiostats and coccidiocidals | Contraceptives |
| Contrast agents | Diabetes therapy |
| Diuretics | Fertility pharmaceuticals |
| Hematinics | Hemostatics |
| Hormone replacement therapy | Immunostimulants |
| Minerals | Muscle relaxants |
| Natural products | Nutraceuticals and nutritionals |
| Obesity therapeutics | Ophthalmic pharmaceuticals |
| Osteoporosis drug | Over the Counter (OTC) pharma |
| Pain therapeutics | Respiratory pharmaceuticals |
| Sedatives and tranquilizers | Transplantation products |
| Urinary acidifiers | Vaccines and adjuvants |
| Vitamins | |

The water-soluble pharmaceuticals useful in the sustained release delivery apparatus according to the present invention include such drugs as peptides, proteins, glycoproteins, polysaccharides, and nucleic acids.

The present invention is particularly appropriate for pharmaceuticals that are very active even in extremely small quantities and whose sustained long-term administration is sought. When used in substantially increased quantities, such pharmaceuticals may be applied to disease and related indications heretofore untreatable over an extended period. The pharmaceuticals may be exemplified by, but not limited to, one or more selected from the group consisting of cytokines (eg. interferons and interleukins), hematopoietic factors (eg. colony-stimulating factors and erythropoietin), cell adhesion factors; immunosuppressants; enzymes (eg. asparaginase, superoxide dismutase, tissue plasminogen activating factor, urokinase, and prourokinase), blood coagulating factors (eg. blood coagulating factor VIII), proteins and peptides including proteins involved in bone metabolism (eg. BMP (bone morphogenetic protein)), antibodies and the like, derivatives thereof and analogues thereof.

The interferons may include alpha, beta, gamma, or any other interferons or any combination thereof. Likewise, the interleukin may be IL-1, IL-2, IL-3, or any others, and the colony-stimulating factor may be multi-CSF (multipotential CSF), GM-CSF (granulocyte-macrophage CSF), G-CSF (granulocyte CSF), M-CSF (macrophage CSF), or any others. Other actives may include vaccine antigens, including live vaccines.

The silicone support material may be of any suitable form. The sustained release support material may take the form of a support matrix or rod, preferably a coated extruded rod structure.

A partially coated rod may be used. Such a structure permits further modification of the release characteristics of the sustained release delivery apparatus according to the present invention. An eccentric or asymmetric rod, optionally partially or fully coated, may be used.

In the process according to the present invention, the silicone support material is formed from a methyl-vinyl siloxane polymer including a fumed silica as reinforcing filler.

A reinforcing filler, in particular a fumed silica, is included in the silicone base polymer. A silicone elastomer including fumed silica sold under the trade designations CS10401 or CS10701, and blends thereof, available from IMMIX Technologies LLC, Cri-Sil Division, have been found to be suitable. A silicone elastomer (and blends thereof) sold under the trade designations CSM 4050-1, PLY-7511 and MED 4104, available from NuSil, have also been found to be suitable.

The silicone base polymer component may be present in amounts of from approximately 15 to 80% by weight, preferably greater than 25% by weight, based on the total weight of the sustained release apparatus. The silicone base polymer can be either liquid form or “gum stock.” Preference is dictated by the type of process used to form and coat the sustained release apparatus. Blending of multiple forms is a typical procedure for obtaining the desired physical properties.

Injection-molding processes may utilize up to 100% liquid silicone base polymer. Compression-molding or transfer-molding may utilise approximately 0.5 to 20% by weight, preferably approximately 2.5 to 7.5% by weight of a liquid silicone component.

The cross-linking agent utilised in the process according to the present invention may be of any suitable type. A siloxane polymer; e.g. a partially methylated polysiloxane polymer, may be used.

In a preferred form the sustained release delivery apparatus may take the form of a kit.

Accordingly, in this aspect of the present invention there is provided a sustained release kit including a plurality of sustained release mini-implants or pellets packaged for delivery in a single treatment,
each mini-implant including
   a silicone support material formed from a methyl-vinyl siloxane polymer including a fumed silica as a reinforcing filler; and
   a pharmaceutically active composition carried in or on the silicone support material;
   the pharmaceutically active composition including
      at least one growth and/or reproduction-associated pharmaceutical; analogue thereof or derivative thereof; and
      a carrier therefor;
each implant being of insufficient size and/or payload individually to provide a predetermined desired threshold blood level of pharmaceutical active for treatment of a selected growth and/or reproduction-associated indication.

Preferably the multiple sustained release mini-implants are packaged in a biodegradable sheath.

Alternatively or in addition the sustained release kit may include at least one sustained release mini tablet implant packaged for delivery in a single treatment,
the or each mini tablet implant including
   a sustained release composition in a unit dosage form including
      approximately 1% to 20% by weight alkali metal chloride;
      approximately 0.5% to 5% by weight lubricant; and
      approximately 75% to 97.5% by weight growth and/or
   reproduction-associated pharmaceutical component;
   a non-silicone pharmaceutical carrier therefor;
the or each implant together being of substantially reduced size and/or payload relative to an equivalent immediate release treatment.

Preferably the or each mini-implant has a payload of approximately 30% to 70% by weight of the total payload of an equivalent immediate release treatment for an equivalent period.

More preferably when a plurality of sustained release mini-implants are used, each implant is of insufficient size and/or payload individually to provide a predetermined required threshold blood level of pharmaceutical active for treatment of a selected indication. For example the mini-implants, in total, may provide, in use, less than the daily equivalent injectable dosage, for example approximately half the daily equivalent injectable dosage.

Preferably the sustained release kit includes approximately 5 to 20 mini-implants. Each mini-implant may have an axial length of approximately 1 to 40 mm, more preferably approximately 1 to 5 mm, most preferably approximately 2 mm.

In a further embodiment of the present invention there is provided a sustained release composition in a unit dosage form including
at least one growth and/or reproduction-associated pharmaceutical component, analogue thereof or derivative thereof; and
a non-silicone pharmaceutical carrier therefor;
wherein said composition includes
   approximately 1% to 20% by weight alkali metal chloride;
   approximately 0.5% to 5% by weight lubricant; and
   approximately 75% to 97.5% by weight growth and/or reproduction-associated pharmaceutical component.

The applicants have surprisingly found that a sustained release composition may be formulated in an effective unit dosage form, e.g. a compressed or extruded tablet/implant form without the necessity to include a silicone component.

The sustained release composition may be utilised alone, or preferably in combination with the sustained release delivery apparatus described above.

The sustained release composition may be included as a further component in the sustained release kit as described above.

The growth and/or reproduction associated pharmaceutical component may be as described above. The pharmaceutical component may be selected from one or more of the group consisting of hormones (eg. growth hormone, e.g. recombinant porcine somatotropin rPST, growth hormone releasing factor, calcitonin, leuteinizing hormone, leuteinizing hormone releasing hormone, and insulin), hormone agonists and antagonists (e.g. LHRH), growth factors (eg. somatomedin, nerve growth factor, neurotrophic factors, fibroblast growth factor, and hepatocyte proliferation factor. A growth hormone, e.g. a natural or synthetic human, porcine, bovine, ovine or like growth hormone may be used. A recombinant porcine somatotropin (rPST) is preferred.

The pharmaceutical carrier may be the same as, or similar to, the pharmaceutical carriers utilised in the preparation of the mini-implants described below.

A water-soluble substance, or a combination of two or more water-soluble substances, is preferred. Sucrose, sodium chloride or a protein or a mixture thereof are preferred carriers. Sodium chloride, a protein or a mixture thereof is particularly preferred.

The sustained release growth composition may take the form of a compressed tablet or extruded rod, optionally a covered rod or tablet. A mini-tablet implant is preferred. A silicone coating may be applied to the tablet or rod, but is not essential.

The compressed tablet formulation may include suitable fillers or excipients as discussed above. A lubricant, such as magnesium stearate, is particularly preferred.

The growth and/or reproduction-associated composition may accordingly include
approximately 5% to 15% by weight sodium chloride;
approximately 0.5% to 5% by weight magnesium stearate; and
approximately 80% to 94.5% by weight recombinant porcine somatotropin.

The pharmaceutical carrier of the sustained release apparatus may be selected to permit release of the pharmaceutically active component over an extended period of time from the composition.

The carrier may include a water-soluble substance. A water-soluble substance is a substance which plays a role of controlling infiltration of water into the inside of the drug dispersion. There is no restriction in terms of the water-soluble substance so long as it is in a solid state (as a form of a preparation) at the body temperature of an animal or human being to which it is to be administered, and a physiologically acceptable, water-soluble substance.

One water-soluble substance, or a combination of two or more water-soluble substances may be used. The water-soluble substance specifically may be selected from one or more of the group consisting of synthetic polymers (eg. polyethylene glycol, polyethylene polypropylene glycol), sugars (eg. sucrose, mannitol, glucose, dextran, sodium chondroitin sulfate), amino acids (eg. glycine and alanine), mineral salts (eg. sodium chloride), organic salts (eg. sodium citrate) and proteins (eg. human γ-globulin, gelatin and collagen and mixtures thereof). A protein, preferably human γ-globulin, or salt, preferably sodium chloride, or mixtures thereof, are preferred.

The pharmaceutical carrier may constitute from approximately 0% to 30% by weight, preferably approximately 5% to 15% by weight based on the total weight of the pharmaceutically active composition.

The sustained release delivery apparatus may include additional carrier or excipients, fillers, plasticisers, binding agents, pigments and stabilising agents.

Suitable fillers may be selected from the group consisting of talc, titanium dioxide, starch, kaolin, cellulose (microcrystalline or powdered) and mixtures thereof.

Where the sustained release delivery apparatus takes the form of a biocompatible article, e.g. an implant, calcium fillers, e.g. calcium phosphate, are particularly preferred.

Suitable binding agents include polyvinyl pyrrolidine, hydroxypropyl cellulose and hydroxypropyl methyl cellulose and mixtures thereof.

In a preferred aspect of the present invention the sustained release delivery apparatus may take the form of a biocompatible article suitable for insertion into the body of an animal to be treated.

The biocompatible article may include a medical instrument, apparatus or prosthetic device, or part thereof.

For example, the biocompatible article may include a catheter, or prosthetic appliance, or medical implant, e.g. for reconstructive, dental or cosmetic surgery. Implant materials for replacing or filling bone or like defects are particularly preferred.

It will be understood that by incorporating a pharmaceutically active composition in or on such biocompatible articles, a sustained therapeutic effect may be achieved at the site of insertion.

For example, growth factors, e.g. nerve growth factors, may be included, for example to assist the healing process, e.g. after surgical procedures.

The sustained release delivery apparatus of the present invention may have a rod-like shape, for example it is selected from circular cylinders, prisms, and elliptical cylinders. When the device will be administered using an injector-type instrument, a circular cylindrical device is preferred since the injector body and the injection needle typically have a circular cylindrical shape, though other shaped objects may be used.

The size of the pharmaceutical formulation of the present invention may, in the case of subcutaneous administration, be relatively small, e.g. ¼ to 1/10 normal size. For example using an injector-type instrument, the configuration may be circular cylindrical, and the cross-sectional diameter in the case is preferably 0.2 to 15 mm, more preferably 1 to 4 mm, and the axial length being preferably approximately 1 to 40 mm, preferably approximately 1 to 30 mm, more preferably approximately 2 to 4 mm.

The thickness of the outer layer should be selected as a function of the material properties and the desired release rate which can be regulated by varying the number of times the extruded or molded rod is coated. The outer layer thickness is not critical as long as the specified functions of the outer layer are fulfilled. The outer layer thickness is preferably 0.05 mm to 3 mm, more preferably 0.05 mm to 0.25 mm, and even more preferably 0.05 mm to 0.1 mm.

Sustained release implants according to the present invention may preferably have a double-layer structure, in order to achieve long-term zero-order release.

The ratio of the axial length of the pharmaceutical formulation to the cross-sectional diameter of the inner layer may, in any case, be one or more and is more preferably two or more and most preferably three or more.

Where a double-layer structure is used, the pharmaceutical-containing inner layer and the drug-impermeable outer layer may be fabricated separately or simultaneously. Silicone is known for swelling with water and being gas-permeable.

A pharmaceutical formulation with an open end at one terminal may be fabricated by dipping one terminal of the pharmaceutical formulation into a solution which dissolves the outer-layer material and drying it, or by coating one terminal end of the pharmaceutical formulation with a cap made from the outer-layer material. In addition, the fabrication may comprise insertion of the inner layer into an outer-layer casing with a closed-end at one terminal, which are separately produced, and also formation of the inner layer in said casing.

In a further aspect of the present invention there is provided a method for the therapeutic or prophylactic treatment of a condition in an animal (including a human) requiring such treatment, or to improve a physiological characteristic of an animal, which method includes
administering to the animal a sustained release composition in a unit dosage form including
at least one growth and/or reproduction-associated pharmaceutical component, analogue thereof or derivative thereof; and
a non-silicone pharmaceutical carrier therefor;
wherein said composition includes
   approximately 1% to 20% by weight alkali metal chloride;
   approximately 0.5% to 5% by weight lubricant; and
   approximately 75% to 97.5% by weight growth and/or reproduction-associated pharmaceutical component.

Preferably the method includes administering to the animal a sustained release delivery apparatus including
a silicone support material;
a pharmaceutically active composition carried in or on the silicone support material;
the pharmaceutically active composition including
   at least one growth and/or reproduction-associated pharmaceutical component; analogue thereof or derivative thereof; and
a carrier therefor.

The method according to this aspect of the present invention is particularly applicable to the treatment of an animal to improve nutritional and/or growth related characteristics. Accordingly, in a preferred embodiment of this aspect of the present invention there is provided a method for the therapeutic or prophylactic treatment of an animal to improve nutritional and/or growth related characteristics, which method includes
administering to the animal
   a sustained release delivery apparatus including
      a silicone support material formed from a methyl-vinyl siloxane polymer including a fumed silica as reinforcing filler; and
      a growth-associated pharmaceutical composition carried in or on the support material including
         at least one growth-associated pharmaceutical component; and
         a carrier therefor;
   the sustained release delivery apparatus exhibiting generally zero order release
administering to the animal at least one sustained release delivery apparatus, the size and/or number thereof being selected to improve at least one growth-associated physiological characteristic.

Applicants have surprisingly found that utilising the sustained release composition, improvement in nutritional and/or growth-related characteristics in an animal may be achieved while reducing or eliminating one or more of the deleterious effects of sustained release treatment encountered in the prior art. For example, the sustained release delivery apparatus may be administered using a weekly, bi-weekly, monthly or up to 6 monthly dosage regimen.

The nutritional and/or growth-related characteristics in which improvement may be made according to this aspect of the present invention include one or more selected from the group consisting of growth rate (including food conversion ratio), carcass quality (including back fat measurement), plasma urea concentrations and plasma glucose levels.

The sustained release composition may take any suitable form as described above. In a preferred embodiment of this aspect of the present invention the delivery apparatus includes one or more mini implants or pellets, as described above.

The number and/or size of the mini implants or pellets may be selected to improve one or more of the characteristics described above.

As stated above, Applicants have surprisingly found that such improvements may be achieved wherein the mini-implants provide, in use, less than the daily equivalent injectable dosage, for example wherein the mini-implants provide, in use, approximately half the daily equivalent injectable dosage. This may represent a significant saving in cost to the user.

In general, the sustained release apparatus may include approximately 5 to 20 mini-implants. Each mini-implant may have an axial length of approximately 1 to 40 mm, more preferably approximately 1 to 5 mm, most preferably approximately 2 mm.

For example, when the animal to be treated is selected from the group consisting of sheep, cattle, goats, horses, camels, pigs, dogs, cats, ferrets, rabbits, marsupials, buffalos, yacks, primates, humans, birds including chickens, geese and turkeys, rodents including rats and mice, fish, reptiles and the like.

In a preferred embodiment, the feed conversion ratio is improved over an extended period of time and backfat reduction is maintained for a further extended period of time.

For example the feed conversion ratio may be improved for at least approximately 7 days and backfat reduction is maintained for at least approximately 14 days.

For example, when the animal is a pig, 7 to 20 mini-implants each having an axial length of approximately 3 mm, are administered.

For example, 2 to 20 3 mm×2 cm, preferably 5 to 20 3 mm×2 cm mini implants may be used.

Alternatively, for pigs, preferably 1 to 20 3 mm×0.2 cm, more preferably 5 to 20 3 mm×0.2 cm mini implants have been found to be suitable.

The growth-associated pharmaceutical component of the pharmaceutical composition according to this aspect of the present invention may be of any suitable type including live vectors and live cells secreting growth hormones as well as RNA and DNA coding for growth hormones. Preferably, the growth-associated pharmaceutical component includes a growth hormone, more preferably at least one exogenous growth hormone selected from homologous, natural or synthetic growth hormones, analogues, derivatives or fragments thereof.

A recombinant growth hormone, e.g. recombinant porcine somatotropin (rPST) is preferred.

The growth-associated pharmaceutical component may alternatively or in addition include other growth hormone and/or factors. Optionally other pharmaceutical components, as described above, may be included.

In a particularly preferred embodiment of the method according to this aspect of the present invention, the sustained release delivery apparatus includes a plurality of sustained release mini-implants or pellets;
each mini-implant including
   a silicone support material formed from a methyl-vinyl siloxane polymer including a fumed silica as reinforcing filler; and
   a pharmaceutically active composition carried in or on the silicone support material;
   the pharmaceutically active composition including
      at least one growth and/or reproduction-associated pharmaceutical; analogue thereof or derivative thereof; and
      a carrier therefor;
each implant being of insufficient size and/or payload individually to provide a predetermined desired threshold blood level of pharmaceutical active for treatment of a selected growth and/or reproduction-associated indication.

The carrier utilised in the growth-associated pharmaceutical composition may be of any suitable type. The carrier may include a salt (NaCl) and/or a protein component as described above. Applicants have surprisingly found that the inclusion of such a component may assist in the performance of the growth associated component, e.g. growth hormone, in vivo. Whilst we do not wish to be restricted by theory, it is postulated that the carrier may assist in maintaining the biological activity and preventing aggregation of the growth hormone in vivo.

The carrier may alternatively or in addition include one or more refolding agents. The refolding agent may be of any suitable type.

The refolding agent may be selected from one or more of the group consisting of urea, anionic surfactants and cationic surfactants. A cationic surfactant is preferred.

The cationic surfactant may include a cation selected from the group consisting of:

Cetyl timethylammonium cations
Cetyl pyriudinium cations
Tetradecyl trimethylammonium cations
Dodecyl trimethylammonium cations
Mixed n-alkyl dimethyl benzyl ammonium cations
N,N-dimethyl-N-[2-[2-[4-(1,1,3,3,-tetramethyl butyl) phenoxy]ethoxy]ethyl] benzenemethanaminium cations
Dodecyldimethylamine oxide
N-lauroylsarcosine sodium salt
N-lauroyl-N-methyltaurine sodium salt
N-lauryl-β-iminodipropionate sodium salt
3-(N,N-Dimethyl laurylammonio) propane sulphonate sodium salt

The method of administration may include subcutaneous, intraperitoneal intramuscular injection, intranasal insertion or indwelling, intrarectal insertion or indwelling, for example as a suppository or utilising oral administration.

In a preferred form, the multiple sustained release mini-implants are packaged in a biodegradable sheath.

The animals to be treated may be selected from mice, rats, sheep, cattle, goats, horses, camels, pigs, dogs, cats, ferrets, rabbits, marsupials, buffalos, yacks, birds, humans, chickens, geese, turkeys, rodents, fish, reptiles and the like.

The method according to the present invention is particularly applicable to larger animals, e.g. cattle, sheep, pigs, dogs and humans where high dosage levels are required to achieve the prerequisite threshold pharmaceutical active blood levels for successful achievement of improved results in growth characteristics and the like.

The present invention will now be more fully described with reference to the accompanying figures and examples. It should be understood, however, that the description following is illustrative only and should not be taken in any way as a restriction on the generality of the invention described above.

### EXAMPLE 1

An A-part of the PST formulation was prepared as follows.

First a platinum masterbatch (Pt MB) was prepared by mixing on a two-roll mill:
7.0 g 60 durometer silicone-base material containing fumed silica filler (base 1)
0.06 g of a platinum catalyst composition

The platinum catalyst composition was diluted 1:3 with silicone fluid.

This completed the A-part of the PST formulation.

A B-part of the PST formulation was then prepared as follows:

First the following were mixed on a two-roll mill:
23.5 g rPST (freeze dried)
1.80 g of Hydride MB (which contained 33% by weight hydride fluid)
5.2 g of silicone fluid
17.5 g 40 durometer silicone base material containing 20% w/w sugar or salt.

Table 1 below gives the amounts of each ingredient used to make each shot:

TABLE 1

| Preparation | | | | | |
|---|---|---|---|---|---|
| No. | B-side | Pre-Mixed Base | EX849 Base | | Pt MB |
| 1 | 3.0 g | 1.10 g 80% | 0 | g | 0.30 g |
| | | w/w Fine Salt | | | |
| 2 | 3.0 g | 1.10 g 80% | 0 | g | 0.30 g |
| | | w/w Fine Salt | | | |
| 3 | 3.0 g | 1.10 g 80% | 0 | g | 0.30 g |
| | | w/w Fine Salt | | | |
| 4 | 3.5 g | 0.64 g 80% | 0.64 | g | 0.35 g |
| | | w/w Fine Salt | | | |
| 5 | 3.5 g | 0.64 g 80% | 0.64 | g | 0.35 g |
| | | w/w Fine Salt | | | |
| 6 | 3.5 g | 0.64 g 80% | 0.64 | g | 0.35 g |
| | | w/w Fine Salt | | | |
| 7 | 3.5 g | 0.32 g 80% | 0.96 | g | 0.35 g |
| | | w/w Fine Salt | | | |
| 8 | 3.5 g | 0.32 g 80% | 0.96 | g | 0.35 g |
| | | w/w Fine Salt | | | |
| 9 | 3.5 g | 0.32 g 80% | 0.96 | g | 0.35 g |
| | | w/w Fine Salt | | | |
| 10 | 3.5 g | 1.28 g 80% | 0 | g | 0.35 g |
| | | w/w Fine Sugar | | | |
| 11 | 3.5 g | 0.64 g 80% | 0.64 | g | 0.35 g |
| | | w/w Coarse Salt | | | |
| 12 | 3.5 g | 0.64 g 80% | 0.64 | g | 0.35 g |
| | | w/w Coarse Salt | | | |
| 13 | 3.5 g | 1.28 g 20% | 0 | g | 0.35 g |
| | | w/w PEPPG | | | |
| 14 | 3.5 g | 1.28 g 20% | 0 | g | 0.35 g |
| | | w/w PEPPG | | | |

Each implant was “cold” compression molded (<20° C.) and subsequently placed in an incubation oven at 70° C. for fifteen minutes. The heat treatment had no apparent effect on the efficacy of the implants. All samples were then dip coated with liquid silicone and dried at 65° C. for 10 minutes. This process of coating with liquid silicone can be repeated numerous times to achieve different release rates.

### EXAMPLE 2

Example 1 was repeated to produce mini implants having the dimensions 3 mm×4 cm and the composition set forth in Table 2 below.

TABLE 2

| | | | |
|---|---|---|---|
| NaCl | PST | NaCl | Silicone |

| | | | | | |
|---|---|---|---|---|---|
| 5% | 122 mg | 18.5 | mg | 229.4 | mg |
| 10% | 121 mg | 37.0 | mg | 210.00 | mg |
| 20% | 110 mg | 68.00 | mg | 153.00 | mg |

### EXAMPLE 3

Mini implants having the composition of various preparations described above were subcutaneously administered to pigs. Whole blood was collected from the animal via the jugular vein daily to day 14 where the animal was sacrificed. Plasma analyses of plasma urea concentration and plasma glucose concentration were conducted utilising standard techniques.

Pigs were monitored daily by measuring feed intake, growth rate and by blood sampling in order to calculate feed conversion ratios, blood urea and glucose levels. Back fat measurements were undertaken by ultrasound at day 15. The results are presented in Tables 3 to 6.

TABLE 3
Plasma Urea Concentrations - mmol/L

| Size | Implant | | | | | | |
|---|---|---|---|---|---|---|---|
| (3 mm Diameter) | (% NaCl) | Pen No | Day 0 | Day 1 | Day 2 | Day 4 | Day 7 |
| 4 × 1 cm | 5 | 7 | 6.8 | 3.9 | 3.6 | 4.6 | 7.1 |
| 4 × 1 cm | 5 | 16 | 4.9 | 3.5 | 4.0 | 4.3 | 5.3 |
| 4 × 1 cm | 5 | 44 | 5.4 | 3.9 | 4.7 | 5.9 | 6.7 |
| 4 × 1 cm | 10 | 2 | 4.9 | 3.7 | 3.5 | 3.5 | 5.9 |
| 4 × 1 cm | 10 | 4 | 5.7 | 3.3 | 3.2 | 3.2 | 5.4 |
| 4 × 1 cm | 10 | 6 | 4.6 | 2.2 | 3.0 | 2.8 | 4.6 |
| 4 × 1 cm | 20 | 8 | 5.8 | 2.5 | 2.8 | 3.6 | 5.0 |
| 4 × 1 cm | 20 | 12 | 4.7 | 2.7 | 2.3 | 2.4 | 5.4 |
| 4 × 1 cm | 20 | 14 | 6.6 | 3.5 | 4.4 | 5.1 | 2.9 |
| | | Mean | 5.5 | 3.2 | 3.5 | 3.9 | 5.4 |
| Size | Implant | Pen No | Day 0 | Day 1 | Day 2 | Day 4 | Day 7 |
| 2 × 2 cm | 5 | 3 | 4.8 | 4.4 | 4.5 | 4.2 | 4.8 |
| 2 × 2 cm | 5 | 5 | 5.0 | 3.8 | 4.4 | 4.6 | 3.7 |
| 2 × 2 cm | 5 | 13 | 5.2 | 4.6 | 4.1 | 3.7 | 5.6 |
| 2 × 2 cm | 10 | 21 | 5.9 | 3.7 | 3.8 | 3.2 | 5.9 |
| 2 × 2 cm | 10 | 26 | 6.4 | 3.8 | 5.0 | 3.2 | 4.7 |
| 2 × 2 cm | 10 | 35 | 6.7 | 5.4 | 5.2 | 4.1 | 5.4 |
| 2 × 2 cm | 20 | 38 | 5.2 | 3.7 | 4.3 | 3.6 | 4.9 |
| 2 × 2 cm | 20 | 40 | 5.6 | 4.6 | 5.8 | 6.0 | 4.3 |
| 2 × 2 cm | 20 | 43 | 6.1 | 4.0 | 5.2 | 5.0 | 3.9 |
| | | Mean | 5.7 | 4.2 | 4.7 | 4.2 | 4.8 |
| 4 × 2 cm | 5 | 23 | 4.6 | 3.4 | 3.8 | 3.0 | 3.2 |
| 4 × 2 cm | 5 | 32 | 4.9 | 3.9 | 3.9 | 4.1 | 5.5 |
| 4 × 2 cm | 5 | 33 | 6.3 | 4.8 | 3.2 | 3.1 | 7.1 |
| 4 × 2 cm | 10 | 37 | 6.9 | 4.8 | 4.1 | 3.4 | 3.8 |
| 4 × 2 cm | 10 | 46 | 4.9 | 3.3 | 3.3 | 2.7 | 4.5 |
| 4 × 2 cm | 20 | 36 | 6.7 | 3.6 | 3.2 | 2.8 | 3.2 |
| | | Mean | 5.7 | 4.0 | 3.6 | 3.2 | 4.5 |
| PST inj | | 17 | 5.2 | 4.1 | 3.7 | 5.6 | 5.5 |
| PST inj | | 18 | 6.6 | 4.5 | 3.3 | 3.3 | 4.7 |
| PST inj | | 24 | 4.5 | 3.9 | 4.0 | 3.7 | 3.8 |
| PST inj | | 25 | 6.8 | 5.2 | 4.4 | 4.8 | 6.5 |
| PST inj | | 27 | 4.4 | 3.1 | 3.5 | 3.3 | 4.4 |
| PST inj | | 29 | 6.4 | 4.5 | 3.9 | 3.8 | 6.8 |
| PST inj | | 30 | 6.3 | 4.3 | 4.2 | 4.0 | 6.6 |
| PST inj | | 31 | 4.7 | 3.1 | 3.1 | 2.8 | 4.9 |
| PST inj | | 47 | 6.9 | 4.8 | 3.7 | 3.8 | 4.6 |
| | | Mean | 5.8 | 4.1 | 3.8 | 3.9 | 5.3 |
| Control | | 1 | 4.6 | 5.8 | 5.3 | 4.8 | 4.7 |
| Control | | 9 | 4.4 | 4.7 | 4.7 | 5.1 | 4.3 |
| Control | | 10 | 8.2 | 8.5 | 8.3 | 8.8 | 8.6 |
| Control | | 11 | 6.6 | 6.7 | 6.6 | 5.3 | 7.0 |
| Control | | 20 | 6.8 | 7.5 | 6.6 | 7.0 | 8.2 |
| Control | | 22 | 6.0 | 6.4 | 7.0 | 6.4 | 6.8 |
| Control | | 34 | 3.9 | 4.6 | 4.9 | 5.0 | 5.6 |
| Control | | 39 | 5.8 | 6.5 | 6.0 | 4.9 | 5.3 |
| Control | | 42 | 5.5 | 5.6 | 6.8 | 6.0 | 6.5 |

| | | | | | |
|---|---|---|---|---|---|
| Mean | 5.7 | 6.2 | 6.2 | 5.9 | 6.3 |

Mean Blood Urea Levels- Comparison with Negative Controls

| | | | | | | | |
|---|---|---|---|---|---|---|---|
| P values | 1 | cm | 0.62 | 0.00001 | 0.00002 | 0.00314 | 0.15210 |
| T test (paired) | 2 | cm | 0.846 | 0.0005 | 0.003 | 0.004 | 0.015 |
| | 2 × 2 | cm | 0.9543 | 0.0014 | 0.0001 | 0.0003 | 0.0426 |
| | PST inj | | 0.97794 | 0.00047 | 0.00001 | 0.00124 | 0.11997 |

TABLE 4
Plasma Glucose - mmol/L

| Size | Implant | | | | | | |
|---|---|---|---|---|---|---|---|
| (3 mm diameter) | (% NaCl) | Pen No | Day 0 | Day 1 | Day 2 | Day 4 | Day 7 |
| 4 × 1 cm | 5 | 7 | 5.4 | 6.7 | 6.0 | 6.8 | 5.0 |
| 4 × 1 cm | 5 | 16 | 5.1 | 5.7 | 5.1 | 5.8 | 4.6 |
| 4 × 1 cm | 5 | 44 | 6.0 | 6.1 | 5.6 | 6.4 | 5.6 |
| 4 × 1 cm | 10 | 2 | 5.8 | 6.8 | 7.8 | 9.8 | 6.5 |
| 4 × 1 cm | 10 | 4 | 5.2 | 6.2 | 6.2 | 6.0 | 5.2 |
| 4 × 1 cm | 10 | 6 | 5.4 | 5.3 | 6.0 | 6.3 | 4.8 |
| 4 × 1 cm | 20 | 8 | 5.8 | 7.6 | 6.7 | 7.2 | 5.6 |
| 4 × 1 cm | 20 | 12 | 5.3 | 7.5 | 7.5 | 8.4 | 5.2 |
| 4 × 1 cm | 20 | 14 | 4.8 | 6.7 | 5.6 | 6.2 | 5.6 |
| | | Mean | 5.4 | 6.5 | 6.3 | 7.0 | 5.4 |
| Size | Implant | Pen No | Day 0 | Day 1 | Day 2 | Day 4 | Day 7 |
| 2 × 2 cm | 5 | 3 | 5.6 | 5.3 | 5.8 | 7.2 | 5.1 |
| 2 × 2 cm | 5 | 5 | 5.3 | 5.7 | 5.4 | 6.0 | 5.0 |
| 2 × 2 cm | 5 | 13 | 5.3 | 6.1 | 5.5 | 6.8 | 5.0 |
| 2 × 2 cm | 10 | 21 | 5.2 | 6.4 | 6.1 | 6.9 | 5.1 |
| 2 × 2 cm | 10 | 26 | 5.4 | 6.4 | 6.9 | 9.2 | 9.0 |
| 2 × 2 cm | 10 | 35 | 6.0 | 6.1 | 6.1 | 7.0 | 6.3 |
| 2 × 2 cm | 20 | 38 | 5.7 | 5.7 | 5.4 | 6.5 | 5.9 |
| 2 × 2 cm | 20 | 40 | 6.6 | 7.2 | 6.1 | 7.2 | 8.8 |
| 2 × 2 cm | 20 | 43 | 5.4 | 6.0 | 5.6 | 5.8 | 5.7 |
| | | Mean | 5.6 | 6.1 | 5.9 | 7.0 | 6.2 |
| 4 × 2 cm | 5 | 23 | 5.7 | 6.4 | 6.0 | 7.6 | 6.5 |
| 4 × 2 cm | 5 | 32 | 5.6 | 6.1 | 6.4 | 5.9 | 5.6 |
| 4 × 2 cm | 5 | 33 | 5.8 | 7.0 | 6.8 | 8.8 | 6.0 |
| 4 × 2 cm | 10 | 37 | 4.8 | 6.2 | 5.7 | 8.0 | 6.4 |
| 4 × 2 cm | 10 | 46 | 5.1 | 5.8 | 6.3 | 6.3 | 5.1 |
| 4 × 2 cm | 20 | 36 | 5.4 | 8.5 | 6.6 | 8.5 | 6.8 |
| | | Mean | 5.4 | 6.7 | 6.3 | 7.5 | 6.1 |
| PST inj | | 17 | 5.0 | 5.0 | 5.0 | 5.5 | 4.9 |
| PST inj | | 18 | 5.2 | 5.9 | 5.7 | 6.2 | 5.1 |
| PST inj | | 24 | 4.9 | 5.2 | 5.0 | 5.4 | 5.0 |
| PST inj | | 25 | 5.4 | 5.2 | 5.9 | 5.8 | 5.0 |
| PST inj | | 27 | 5.4 | 4.5 | 5.3 | 5.8 | 4.9 |
| PST inj | | 29 | 4.8 | 5.5 | 5.5 | 6.0 | 5.1 |
| PST inj | | 30 | 5.4 | 5.5 | 5.7 | 6.3 | 5.3 |
| PST inj | | 31 | 5.6 | 6.0 | 6.0 | 6.5 | 5.1 |
| PST inj | | 47 | 5.1 | 5.5 | 5.6 | 5.7 | 5.8 |
| | | Mean | 5.2 | 5.3 | 5.5 | 5.9 | 5.1 |
| Control | | 1 | 5.1 | 4.6 | 4.8 | 5.6 | 5.9 |
| Control | | 9 | 5.3 | 5.1 | 5.1 | 6.3 | 5.1 |
| Control | | 10 | 5.3 | 5.4 | 5.4 | 5.3 | 5.3 |
| Control | | 11 | 5.9 | 5.4 | 5.2 | 4.8 | 5.3 |
| Control | | 20 | 4.7 | 4.6 | 5.1 | 4.8 | 4.8 |
| Control | | 22 | 4.8 | 4.6 | 4.8 | 5.4 | 4.9 |
| Control | | 34 | 5.9 | 5.4 | 5.4 | 5.7 | 5.2 |
| Control | | 39 | 5.0 | 5.1 | 4.9 | 5.2 | 5.1 |
| Control | | 42 | 5.5 | 5.4 | 5.3 | 5.4 | 7.8 |

| | | | | | |
|---|---|---|---|---|---|
| Mean | 5.3 | 5.0 | 5.1 | 5.4 | 5.5 |

TABLE 5
PST Backfat Measurements at day 15 (P2 (mm))

| Controls | | PST | | 4 × cm | | 1 cm | | 2 cm | |
|---|---|---|---|---|---|---|---|---|---|
| 1 | 14 | 17 | 9 | 23 | 11 | 2 | 11 | 3 | 12.5 |
| 9 | 15.5 | 18 | 12 | 32 | 11.5 | 4 | 9.5 | 5 | 10.5 |
| 10 | 15 | 24 | 10.5 | 33 | 9.5 | 6 | 9 | 13 | 12.5 |
| 11 | 14.5 | 25 | 13.5 | 37 | 11.5 | 8 | 10 | 21 | 12 |
| 20 | 14.5 | 27 | 11.5 | 46 | 13 | 12 | 12 | 26 | 13 |
| 22 | 12.5 | 29 | 12.5 | | | 14 | 14 | 35 | 13.5 |
| 34 | 10 | 30 | 12.5 | | | 7 | 13 | | |
| 39 | 14 | 31 | 10 | | | 16 | 13.5 | | |
| 42 | 11 | 47 | 10.5 | | | | | | |
| Mean | 13.4 | | 11.3 | | 11.3 | | 11.5 | | 12.3 |
| SD | 1.9 | | 1.4 | | 1.25 | | 1.91 | | 1.03 |
| | P value | | 0.016 | | 0.04 | | 0.05 | | 0.21 |

Compare all implanted pigs with negative controls, mean backfat is 11.6 mm and p value=0.01

Compare each implanted group with positive controls p value>0.05

TABLE 6
Feed Conversion Ratios

| Day 0 to Day 7- 1 cm | | | | Day 0 to Day 14- 1 cm | | | |
|---|---|---|---|---|---|---|---|
| Pen | | Weight | | Pen | | Weight | |
| No | Feed | gain | FCR | No | Feed | gain | FCR |
| 7 | 13.54 | 5.6 | 2.42 | 7 | 26.66 | 6.8 | 3.92 |
| 16 | 14.63 | 5.2 | 2.81 | 16 | 29.6 | 10.6 | 2.79 |
| 44 | 15.99 | 6 | 2.67 | 44 | 32.8 | 11.8 | 2.78 |
| 2 | 18.95 | 9.4 | 2.02 | 2 | 38.87 | 14.8 | 2.63 |
| 4 | 15.79 | 8.8 | 1.79 | 4 | 34.59 | 15.8 | 2.19 |
| 6 | 13.54 | 6.4 | 2.12 | 6 | 28.77 | 11 | 2.62 |
| 8 | 17.32 | 9 | 1.92 | 8 | 36.65 | 13.6 | 2.69 |
| 12 | 16.72 | 7.6 | 2.20 | 12 | 39.28 | 14.6 | 2.69 |
| 14 | 11.63 | 3.8 | 3.06 | 14 | 22.67 | 5.8 | 3.91 |
| Mean | 15.35 | 6.87 | 2.33 | Mean | 32.21 | 11.64 | 2.91 |
| Day 0 to Day 7 - 2 cm | | | | Day 0 to Day 14 - 2 cm | | | |
| Pen | | Weight | | | | Weight | |
| No | Feed | gain | FCR | Pen No | Feed | gain | FCR |
| 3 | 13.21 | 6.2 | 2.13 | 3 | 27.23 | 11.8 | 2.31 |
| 5 | 11.57 | 4.2 | 2.75 | 5 | 22.44 | 7 | 3.21 |
| 13 | 15.65 | 7.4 | 2.11 | 13 | 33.26 | 12.4 | 2.68 |
| 21 | 12.71 | 7.2 | 1.77 | 21 | 29.33 | 12.8 | 2.29 |
| 26 | 14.46 | 8.4 | 1.72 | 26 | 32.43 | 14.2 | 2.28 |
| 35 | 16.35 | 6.4 | 2.55 | 35 | 33.13 | 12.8 | 2.59 |
| 38 | 16 | 6.4 | 2.50 | 38 | 33.2 | 14.2 | 2.34 |
| 40 | 21.65 | 10.6 | 2.04 | 40 | 45.05 | 17.2 | 2.62 |
| 43 | 16.27 | 6.6 | 2.47 | 43 | 30.33 | 9.6 | 3.16 |
| Mean | 15.32 | 7.04 | 2.23 | Mean | 31.82 | 12.44 | 2.61 |
| Day 0 to Day 7 - 4 × 2 cm | | | | Day 0 to Day 14 - 4 × 2 cm | | | |
| Pen | | Weight | | | | Weight | |
| No | Feed | gain | FCR | Pen No | Feed | gain | FCR |
| 23 | 19.87 | 9 | 2.21 | 23 | 40.97 | 17.2 | 2.38 |
| 32 | 19 | 8.4 | 2.26 | 32 | 38.35 | 16.4 | 2.34 |
| 33 | 18.89 | 10.2 | 1.85 | 33 | 36.99 | 15.4 | 2.40 |
| 37 | 17.69 | 9.2 | 1.92 | 37 | 35.87 | 15 | 2.39 |
| 46 | 15.64 | 8.8 | 1.78 | 46 | 35.05 | 15 | 2.34 |
| 36 | 16.46 | 7.8 | 2.11 | 36 | | | |
| Mean | 17.925 | 8.90 | 2.02 | Mean | 37.45 | 15.80 | 2.37 |
| Day 0 to Day 7 -PST Injection | | | | Day 0 to Day 14 -PST Injection | | | |
| Pen | | Weight | | Pen | | Weight | |
| No | Feed | gain | FCR | No | Feed | gain | FCR |
| 17 | 14.61 | 5.4 | 2.71 | 17 | 30.61 | 14 | 2.19 |
| 18 | 12.81 | 5.6 | 2.29 | 18 | 25.28 | 11.6 | 2.18 |
| 24 | 17.9 | 10.8 | 1.66 | 24 | 38.25 | 20.2 | 1.89 |
| 25 | 15.93 | 7.8 | 2.04 | 25 | 30.6 | 14.2 | 2.15 |
| 27 | 14.67 | 7.2 | 2.04 | 27 | 32.36 | 17.4 | 1.86 |
| 29 | 16.7 | 9.4 | 1.78 | 29 | 32.71 | 16.2 | 2.02 |
| 30 | 17.28 | 9.2 | 1.88 | 30 | 34.14 | 18.8 | 1.82 |
| 31 | 17.56 | 7.2 | 2.44 | 31 | 35.54 | 16.6 | 2.14 |
| 47 | 15.96 | 7.8 | 2.05 | 47 | 30.45 | 13.8 | 2.21 |
| Mean | 15.94 | 7.82 | 2.10 | Mean | 32.22 | 15.87 | 2.05 |
| Day 0 to Day 7 - Controls | | | | Day 0 to Day 14 - Controls | | | |
| Pen | | Weight | | | | Weight | |
| No | Feed | gain | FCR | Pen No | Feed | gain | FCR |
| 1 | 15.36 | 4.6 | 3.34 | 1 | 32.66 | 11.8 | 2.77 |
| 9 | 15.71 | 7.2 | 2.18 | 9 | 32.31 | 13.4 | 2.41 |
| 10 | 21.22 | 6.4 | 3.32 | 10 | 43.28 | 14.2 | 3.05 |
| 11 | 20.82 | 7.4 | 2.81 | 11 | 42.46 | 13.6 | 3.12 |
| 20 | 20.92 | 7.8 | 2.68 | 20 | 41.24 | 14.4 | 2.86 |
| 22 | 20.89 | 8.6 | 2.43 | 22 | 42.71 | 16.6 | 2.57 |
| 34 | 19.13 | 8 | 2.39 | 34 | 39.82 | 18 | 2.21 |
| 39 | 16.91 | 3.4 | 4.97 | 39 | 33.14 | 10.2 | 3.25 |
| 42 | 19.26 | 7.6 | 2.53 | 42 | 36.32 | 15 | 2.42 |
| Mean | 18.91 | 6.78 | 2.96 | Mean | 38.22 | 14.13 | 2.74 |

New formulations allowing the controlled release have been developed based on the number of liquid silicone coatings. These are shown in Table 7.

TABLE 7
New formulations for PST - in vitro release data for 1 cm × 4
implants (3 mm diameter). Amount of rPST released per day (mg).

| | Day 2 | Day 3 | Day 4 | Day 7 | Day 9 | Day 14 |
|---|---|---|---|---|---|---|
| 15% NaCl 1 coat silicone | 1.857 | 0.961 | 2.669 | 4.236 | 5.23 | 4.15 |
| 10% NaCl 1 coat silicone | 1.919 | 1.218 | 3.382 | 5.369 | 6.628 | 5.26 |
| 5% NaCl 1 coat silicone | 1.379 | 0.354 | 0.984 | 1.562 | 1.929 | 1.531 |
| 10% NaCl 2 coats silicone | 1.302 | 0.231 | 0.642 | 1.019 | 1.258 | 0.998 |
| 10% NaCl 3 coats silicone | 1.534 | | | | | |
| 15% mannitol 1 coat silicone | 1.981 | 0.879 | 2.44 | 3.873 | 4.782 | 3.795 |
| 10% mannitol 1 coat silicone | 1.703 | 0.457 | 1.27 | 2.016 | 2.486 | 1.975 |
| 5% mannitol 1 coat silicone | 0.917 | 0.056 | 0.156 | 0.258 | 0.307 | 0.2043 |
| 10% mannitol 2 coats silicone | 1.657 | 0.097 | 0.271 | 0.43 | 0.53 | 0.421 |
| 10% mannitol 3 coats silicone | 1.672 | 0.231 | 0.642 | 1.019 | 1.258 | 0.998 |
| 5% NaCl 5% mannitol 1 coat | 2.058 | 0.334 | 0.927 | 1.472 | 1.817 | 1.442 |
| 5% NacCl 10% mannitol 1 coat | 2.906 | 0.93 | 2.583 | 4.1 | 5.062 | 4.017 |
| 10% NaCl 5% mannitol 1 coat | 3.029 | 0.961 | 2.669 | 4.236 | 5.23 | 4.14 |
| 7.5% NaCl 7.5% mannitol 1 coat | 2.674 | 0.93 | 2.583 | 4.1 | 5.062 | 4.017 |
| 7.5% NaCl 7.5% mannitol 2 coats | 1.749 | 0.57 | 1.584 | 2.514 | 3.104 | 2.463 |
| 7.5% NaCl 7.5% mannitol 3 coats | 1.873 | 0.159 | 0.442 | 0.702 | 0.866 | 0.687 |

### EXAMPLE 4

Laboratory-scale formulation of compressed tablet implants of recombinant porcine somatotropin (rPST).

The tableting procedure was as follows:
the “base-formulation” was weighed into a polyethylene terephthalate container (polyethylene lid), and the weight recorded;
the requisite amount of magnesium stearate was calculated and weighed into the polyethylene terephthalate container;
the formulation was mixed by tumbling for ca. 15 minutes;
tablets were prepared (details below); and
subsequent to tableting (described below), the tablets were placed in polyethylene sample vials, sealed, labelled (with the sample number, study number, type of sample, date collected, and storage conditions) and placed in storage (4° C.).

The tableting protocol involved:
filling the tableting die cavity with powder;
compression of the powder;
repeat of the above steps until the requisite loading (ca. 5, 10, 30, 40, 60 and 70 mg) was achieved;
ejection of the full tablet (or parts thereof) from the die cavity by raising the lower punch.
Pressing pressure: ca 1200 psi
Conditions: Temperature=20° C.
   Humidity=ambient
      Tablet Properties:
Dimension: nominal 2.95 mm diameter×length (in mm) as required
Mass per tablet: nominal 5 mg per 1.0 mm tablet

Sodium chloride (NaCl) is finely ground utilising a mortar and pestle prior to tableting.

Details of the tablet batches are provided in Table 8.

TABLE 8

| | rPST − NaCl | | |
|---|---|---|---|
| Batch | mass (g) | Mg stearate | |
| ID | (% rPST − NaCl) | mass (g) | Tablet data |
| 1 | 2.217 (97.3) | 0.062 | 154 tablets |
| | Smart Tab M | | average length = |
| | | | 3 mm/tablet |
| | | | average mass = |
| | | | 14.8 mg/tablet |
| | | | Pure rPST 13 mg/tablet |
| 2 | 2.325 (97.3) | 0.065 | 144 tablets |
| | Smart Tab A | | average length = |
| | | | 3.4 mm/tablet |
| | | | average mass = |
| | | | 16.6 mg/tablet |
| | | | Pure rPST 13 mg/tablet |
| | | | (PST only 90% pure) |

A number of the compressed tablets were implanted via sub-cutaneous injection in pigs. The results illustrating improved feed conversion efficiency, fat reduction, etc are shown in Table 9.

TABLE 9

| | | | | 0-7 days | | |
|---|---|---|---|---|---|---|
| | | | | Feed | Weight | |
| | No of | Implant | | intake | increase | |
| | pigs | size PST | | (kgs) | (kgs) | FCR |
| Group 1 | 6 | 5 | mg/day | 16.33 | 8.30 | 1.97 |
| PST Injection A | | | | | | |
| Group 2 | 6 | 5 | mg/day | 16.78 | 9.43 | 1.78 |
| PST Injection M | | | | | | |
| Group 8 | 6 | — | | 17.18 | 6.03 | 2.85 |
| Sham Control | | | | | | |
| Group 4 | 6 | 13 | mg | 13.95 | 7.53 | 1.85 |
| Smart Tab M | | | | | | |
| | | 3 × per week | | | | |
| Group 5 | 6 | 14 | mg | 16.77 | 8.00 | 2.10 |
| Smart Tab A | | | | | | |

3 × per week

### EXAMPLE 5

The pig experiments illustrated in Example 4 were repeated over 7, 14 and 21 days with varying numbers of implants.

The results are shown in Tables 10 and 11.

TABLE 10

0-7 days

| | | | | Feed | Weight | | | |
|---|---|---|---|---|---|---|---|---|
| | No of | | Implant | intake | increase | | | P2 mm |
| | pigs | Days | size PST | (kgs) | (kgs) | FCR | P2 mm | change |
| Group 4 | 6 | 0-7 | 3 × 13 mg | 13.95 | 7.53 | 1.85 | 10.2 | −0.1 |
| Smart Tab M | | | | | | | | |
| Group 5 | 6 | 0-7 | 3 × 14 mg | 16.77 | 8.00 | 2.10 | 11.0 | +0.8 |
| Smart Tab A | | | | | | | | |
| Group 8 | 6 | | — | 17.18 | 6.03 | 2.85 | 12.2 | +0.9 |
| Sham Control | | | | | | | | |
| | | | | 7-14 days | | | | |
| | | | | Feed | Weight | | | |
| | No of | | Implant | intake | increase | | | P2 mm |
| | pigs | Days | size PST | (kgs) | (kgs) | FCR | P2 mm | change |
| Group 4 | 6 | 7-14 | 1 × 6.5 mg | 14.59 | 4.53 | 2.69 | 10.7 | +0.5 |
| Smart Tab M | | | | | | | | |
| Group 5 | 6 | 7-14 | 3 × 14 mg | 17.68 | 7.27 | 2.43 | 12.2 | +1.2 |
| Smart Tab A | | | | | | | | |
| Group 8 | 6 | | — | 18.10 | 6.63 | 2.73 | 12.9 | +0.7 |
| Sham Control | | | | | | | | |
| | | | | 14-21 days | | | | |
| | | | | Feed | Weight | | | |
| | No of | | Implant | intake | increase | | | P2 mm |
| | pigs | Days | size PST | (kgs) | (kgs) | FCR | P2 mm | change |
| Group 4 | 6 | 14-21 | 1 × 13 mg | 16.75 | 6.97 | 2.40 | 11.3 | +0.6 |
| Smart Tab M | | | | | | | | |
| Group 5 | 6 | 14-21 | 3 × 14 mg | 19.50 | 7.47 | 2.61 | 12.1 | −0.1 |
| Smart Tab A | | | | | | | | |
| Group 8 | 6 | | — | 18.64 | 7.00 | 2.66 | 13.1 | +0.2 |
| Sham Control | | | | | | | | |

TABLE 11

| | | | | 0-21 days | | | | |
|---|---|---|---|---|---|---|---|---|
| | | | | Feed | Weight | | | |
| | No of | | Implant | intake | increase | | | P2 mm |
| | pigs | Days | size PST | (kgs) | (kgs) | FCR | P2 mm | change |
| Group 4 | 6 | 0-7 | 3 × 13 mg | 45.30 | 18.27 | 2.51 | 11.3 | +1.0 |
| Smart Tab M | | 7-14 | 1 × 1.6 mg | | | | | |
| | | 14-21 | 1 × 13 mg | | | | | |
| Group 5 | 6 | 0-7 | 3 × 14 mg | 53.91 | 22.73 | 2.37 | 12.1 | +1.8 |
| Smart Tab A | | 7-14 | 3 × 14 mg | | | | | |
| | | 14-21 | 3 × 14 mg | | | | | |
| Group 8 | 6 | — | — | 53.91 | 19.67 | 2.74 | 13.1 | +1.8 |
| Sham Control | | | | | | | | |

Surprisingly, for the Smart Tab M formulation, the feed conversion ratio utilising a single 13 mg implant is approximately equivalent to the daily injection regimen.

The best fat reduction (as measured by P2) is achieved utilising the Smart Tab M formulation.

### EXAMPLE 6

Study Location: Pig Research and Training Centre (PRTC)
Department of Primary Industries
600 Sneydes Rd
Werribee VIC, Australia.

Mini-implants in the form of co-extruded covered rods and having the compositions set out in Table 12 below were implanted via sub-cutaneous injection in 58 Male Large White Landrace Cross Pigs.

The groups and treatment allocation for Part A of the Trial are set out in Table 13 below.

The Schedule of Events for the pig trials are set out below.

The results achieved are set out in Tables 14 to 17 below.

Tables 15 to 18 illustrate the effect of varying the length/number of implants on feed conversion efficiency and fat reduction.

TABLE 12
Formulations - All 3 mm diameter covered rod

| | | | Human Gamma- |
|---|---|---|---|
| Implant Name | PST | NaCl | Globulin |
| 1a | 20% | 5% | 15% |
| 2a | 20% | 5% | 0% |
| 3a | 20% | 10% | 15% |
| 4a | 20% | 10% | 0% |
| 5a | 20% | 0% | 15% |

Schedule of Events

The trial was divided into 2 parts—Part A and Part B.

### Part A

| | | |
|---|---|---|
| 19/4/04 | Day 1-7 | Pigs were selected on the basis of bodyweight and |
| | | assigned to treatment groups. |
| | | Pig weights were variable between groups. |
| | | Pigs grouped into 5 groups of 10 and 2 groups of 5. |
| | | Within each group - pigs similar weight. |
| | | Pigs moved to experimental grower shed. |
| | | Pigs weighed. |
| | | Pigs ear tagged. |
| | | All pigs fed ad libitum |

TABLE 13
Groups and Treatment Allocation

| Pig No | Group No | Treatment | Pig No | Group No | Treatment |
|---|---|---|---|---|---|
| 1 | 1 | 20 × 0.2 | 31 | 4 | 20 × 0.2 |
| 2 | 1 | 20 × 0.2 | 32 | 4 | 20 × 0.2 |
| 3 | 1 | 20 × 0.2 | 33 | 4 | 20 × 0.2 |
| 4 | 1 | 20 × 0.2 | 34 | 4 | 20 × 0.2 |
| 5 | 1 | 20 × 0.2 | 35 | 4 | 20 × 0.2 |
| 6 | 1 | 10 × 0.4 | 36 | 4 | 10 × 0.4 |
| 7 | 1 | 10 × 0.4 | 37 | 4 | 10 × 0.4 |
| 8 | 1 | 10 × 0.4 | 38 | 4 | 10 × 0.4 |
| 9 | 1 | 10 × 0.4 | 39 | 4 | 10 × 0.4 |
| 10 | 1 | 10 × 0.4 | 40 | 4 | 10 × 0.4 |
| 11 | 2 | 20 × 0.2 | 41 | 5 | 20 × 0.2 |
| 12 | 2 | 20 × 0.2 | 42 | 5 | 20 × 0.2 |
| 13 | 2 | 20 × 0.2 | 43 | 5 | 20 × 0.2 |
| 14 | 2 | 20 × 0.2 | 44 | 5 | 20 × 0.2 |
| 15 | 2 | 20 × 0.2 | 45 | 5 | 20 × 0.2 |
| 16 | 2 | 10 × 0.4 | 46 | 5 | 10 × 0.4 |
| 17 | 2 | 10 × 0.4 | 47 | 5 | 10 × 0.4 |
| 18 | 2 | 10 × 0.4 | 48 | 5 | 10 × 0.4 |
| 19 | 2 | 10 × 0.4 | 49 | 5 | 10 × 0.4 |
| 20 | 2 | 10 × 0.4 | 50 | 5 | 10 × 0.4 |
| 21 | 3 | 20 × 0.2 | 51 | 6 | Negative |
| 22 | 3 | 20 × 0.2 | 52 | 6 | Negative |
| 23 | 3 | 20 × 0.2 | 53 | 6 | Negative |
| 24 | 3 | 20 × 0.2 | 54 | 6 | Negative |
| 25 | 3 | 20 × 0.2 | 55 | 7 | PST inj |
| 26 | 3 | 10 × 0.4 | 56 | 7 | PST inj |
| 27 | 3 | 10 × 0.4 | 57 | 7 | PST inj |
| 28 | 3 | 10 × 0.4 | 58 | 7 | PST inj |
| 29 | 3 | 10 × 0.4 | | | |
| 30 | 3 | 10 × 0.4 | | | |

Group Implant name
1→1a
2→2a
3→3a
4→4a
5→5a

For each of the treated groups 5 pigs were treated with 20×0.2 cm implants and 5 with 10×0.4 cm implants.

| | | |
|---|---|---|
| 20/4/04 | Day −6 | Record feed refusals and feed offered. |
| | | Check pigs eating - know how to use flaps and |
| | | drinkers. |
| 21/4/04 | Day −5 | Record feed refusals and feed offered. |
| | | Check pigs eating - know how to use flaps and |
| | | drinkers. |
| 22/4/04 | Day −4 | Record feed refusals and feed offered. |
| | | Check pigs eating - know how to use flaps and |
| | | drinkers. |
| 23/4/04 | Day −3 | Record feed refusals and feed offered. |
| | | Check pigs eating - know how to use flaps and |
| | | drinkers. |
| 24/4/04 | Day −2 | Record feed refusals and feed offered. |
| | | Check pigs eating - know how to use flaps and |
| | | drinkers. |
| 25/4/04 | Day −1 | Record feed refusals and feed offered. |
| | | Check pigs eating - know how to use flaps and |
| | | drinkers. |
| 26/4/04 | Day 0 | Record feed refusals and feed offered. |
| | | Weigh pigs. |
| | | Implant pigs according to treatment allocations. |
| | | Inject PST positive control pigs as per standard |
| | | treatment. |
| | | Measure and record P2 (backfat). |
| 27/4/04 | Day 1 | Record feed refusals and feed offered |
| | | Inject PST positive control pigs as per standard |
| | | treatment. |
| 28/4/04 | Day 2 | Record feed refusals and feed offered |
| | | Inject PST positive control pigs as per standard |
| | | treatment. |
| 29/4/04 | Day 3 | Record feed refusals and feed offered |
| | | Inject PST positive control pigs as per standard |
| | | treatment. |
| 30/4/04 | Day 4 | Record feed refusals and feed offered |
| | | Inject PST positive control pigs as per standard |
| | | treatment. |
| | | Weigh pigs. |
| | | Calculate Feed Conversion Ratio (FCR). |
| 1/5/04 | Day 5 | Record feed refusals and feed offered |
| | | Inject PST positive control pigs as per standard |
| | | treatment. |
| 2/5/04 | Day 6 | Record feed refusals and feed offered |
| | | Inject PST positive control pigs as per standard |
| | | treatment. |
| 3/5/04 | Day 7 | Record feed refusals and feed offered |
| | | Inject PST positive control pigs as per standard |
| | | treatment. |
| | | Weigh pigs. |
| | | Calculate FCR. |
| 4/5/04 | Day 8 | Record feed refusals and feed offered |
| | | Inject PST positive control pigs as per standard |
| | | treatment. |
| 5/5/04 | Day 9 | Record feed refusals and feed offered |
| | | Inject PST positive control pigs as per standard |
| | | treatment. |
| 6/5/04 | Day 10 | Record feed refusals and feed offered |
| | | Inject PST positive control pigs as per standard |
| | | treatment. |
| | | Weigh all pigs. |
| | | Calculate FCR. |
| 7/5/04 | Day 11 | Record feed refusals and feed offered |
| | | Inject PST positive control pigs as per standard |
| | | treatment. |
| 8/5/04 | Day 12 | Record feed refusals and feed offered |
| | | Inject PST positive control pigs as per standard |
| | | treatment. |
| 9/5/04 | Day 13 | Record feed refusals and feed offered |
| | | Inject PST positive control pigs as per standard |
| | | treatment. |
| 10/5/04 | Day 14 | Record feed refusals and feed offered |
| | | Inject PST positive control pigs as per standard |
| | | treatment. |
| | | Weigh all pigs. |
| | | Measure P2. |
| | | Calculate FCR. |

TABLE 14

| | | | | | Weight | | Improvement | |
|---|---|---|---|---|---|---|---|---|
| | | | | Daily | Change | Feed | over | |
| Implant | | | Length/No. | Equiv | kg Day | Conversion | untreated | P2 (mm) |
| name | Treatment | | of Implants | Dose (mg) | 0-7 | Ratio | Control | Day 7 |
| 1a | 20% | PST | 0.2 cm × 20 | 8.6 | 8.0 | 2.29 | 10.1% | 10.3 |
| | 5% | Salt | | | | | | |
| | 15% | Protein | | | | | | |
| 2a | 20% | PST | 0.2 cm × 20 | 8.6 | 8.8 | 2.08 | 18.4% | 10.1 |
| | 5% | Salt | | | | | | |
| 3a | 20% | PST | 0.2 cm × 20 | 8.6 | 8.2 | 2.53 | 0% | 11.9 |
| | 10% | Salt | | | | | | |
| | 15% | Protein | | | | | | |
| 4a | 20% | PST | 0.2 cm × 20 | 8.6 | 10.2 | 1.96 | 23.1% | 11.7 |
| | 10% | Salt | | | | | | |
| 5a | 20% | PST | 0.2 cm × 20 | 8.6 | 9.9 | 2.08 | 18.4% | 10.9 |
| | 15% | Protein | | | | | | |

| | | | | | | |
|---|---|---|---|---|---|---|
| Daily | — | 6.0 | 9.3 | 2.09 | 18.0% | 11.1 |
| Injection | | | | | | |
| Control | — | — | 9.5 | 2.55 | — | 14.5 |

TABLE 15

| | | | | | Weight | | Improvement | |
|---|---|---|---|---|---|---|---|---|
| | | | | Daily | Change kg | Feed | over | |
| Implant | | | Length/No. | Equiv | Day | Conversion | untreated | P2 (mm) |
| Name | Treatment | | of Implants | Dose (mg) | 0-7 | Ratio | Control | Day 7 |
| 1a | 20% | PST | 0.4 cm × 10 | 8.6 | 7.6 | 2.30 | 9.8% | 10.5 |
| | 5% | Salt | | | | | | |
| | 15% | Protein | | | | | | |
| 2a | 20% | PST | 0.4 cm × 10 | 8.6 | 7.6 | 2.64 | −3.5% | 10.1 |
| | 5% | Salt | | | | | | |
| 3a | 20% | PST | 0.4 cm × 10 | 8.6 | 8.2 | 2.56 | 0% | 11.9 |
| | 10% | Salt | | | | | | |
| | 15% | Protein | | | | | | |
| 4a | 20% | PST | 0.4 cm × 10 | 8.6 | 8.8 | 2.16 | 15.3% | 10.7 |
| | 10% | Salt | | | | | | |
| 5a | 20% | PST | 0.4 cm × 10 | 8.6 | 9.1 | 2.36 | 7.5% | 12.9 |
| | 15% | Protein | | | | | | |

| | | | | | | |
|---|---|---|---|---|---|---|
| Daily | — | 6.0 | 9.3 | 2.09 | 18.0% | 11.1 |
| Injection | | | | | | |
| Control | — | — | 9.5 | 2.55 | — | 14.5 |

TABLE 16

| | | | | Daily | | | Improvement | |
|---|---|---|---|---|---|---|---|---|
| | | | | Equiv | Weight | Feed | over | |
| Implant | | | Length/No. | Dose | Change kg | Conversion | untreated | P2 (mm) |
| Name | Treatment | | of Implants | (mg) | Day 0-10 | Ratio | Control | Day 14 |
| 1a | 20% | PST | 0.2 cm × 20 | 6 | 11.3 | 2.21 | 10.9% | 11.2 |
| | 5% | Salt | | | | | | |
| | 15% | Protein | | | | | | |
| 2a | 20% | PST | 0.2 cm × 20 | 6 | 12.0 | 2.17 | 12.5% | 10.6 |
| | 5% | Salt | | | | | | |
| 3a | 20% | PST | 0.2 cm × 20 | 6 | 10.8 | 2.79 | −12.5% | 13.0 |
| | 10% | Salt | | | | | | |
| | 15% | Protein | | | | | | |
| 4a | 20% | PST | 0.2 cm × 20 | 6 | 13.6 | 2.13 | 14.1% | 12.5 |
| | 10% | Salt | | | | | | |
| 5a | 20% | PST | 0.2 cm × 20 | 6 | 13.4 | 2.24 | 9.7% | 11.7 |
| | 15% | Protein | | | | | | |

| | | | | | | |
|---|---|---|---|---|---|---|
| Daily | — | 6 | 14.0 | 1.94 | 21.8% | 13.5 |
| Injection | | | | | | |
| Control | — | — | 13.6 | 2.48 | — | 14.9 |

TABLE 17

| | | | | Daily | | | Improvement | |
|---|---|---|---|---|---|---|---|---|
| | | | | Equiv | Weight | Feed | over | |
| Implant | | | Length/No. | Dose | Change kg | Conversion | untreated | P2 (mm) |
| Name | Treatment | | of Implants | (mg) | Day 0-10 | Ratio | Control | Day 14 |
| 1a | 20% | PST | 0.4 cm × 10 | 6 | 11.6 | 2.18 | 12.1% | 10.3 |
| | 5% | Salt | | | | | | |
| | 15% | Protein | | | | | | |
| 2a | 20% | PST | 0.4 cm × 10 | 6 | 11.3 | 2.36 | 4.9% | 10.6 |
| | 5% | Salt | | | | | | |
| 3a | 20% | PST | 0.4 cm × 10 | 6 | 12.4 | 2.43 | 2.0% | 12.4 |
| | 10% | Salt | | | | | | |
| | 15% | Protein | | | | | | |
| 4a | 20% | PST | 0.4 cm × 10 | 6 | 11.4 | 2.39 | 3.6% | 12.9 |
| | 10% | Salt | | | | | | |
| 5a | 20% | PST | 0.4 cm × 10 | 6 | 13.4 | 2.34 | 4.0% | 13.3 |
| | 15% | Protein | | | | | | |

| | | | | | | |
|---|---|---|---|---|---|---|
| Daily | — | 6 | 14.0 | 1.94 | 21.8% | 13.5 |
| Injection | | | | | | |
| Control | — | — | 13.6 | 2.48 | — | 14.9 |

Table 14 illustrates that best results were achieved with Implants 2a and 4a (20% PST and 5% or 10% Salt (NaCl).

Table 15 illustrates that superior results are achieved with the combination 0.2 cm×20 Length/Number of Implants, despite the fact that the nominal daily equivalent dose with the combination 0.4 cm×10 is the same (8.6 mg in each case).

Table 16 illustrates that whilst there is a decrease in food conversion efficiency in days 7 to 14, reduction in the backfat (P2 (mm)) persists up to day 14.

Table 17 duplicates the findings of Table 16 for feed conversion efficiency, but the reduction in backfat (P2 (mm)) is similar to that achieved for both Length/Number of Implant combinations.

### Part B

Pigs used for Part A were re-implanted for Part B using a different treatment schedule as set out in Table 18 below.

TABLE 18
Allocations and Treatments
Part B

| Pig Numbers | Treatment |
|---|---|
| 1, 2, 3, 4, 5 | 7 × 02. cm − 5a |
| 11, 12, 13, 14, 15 | 14 × 0.2 cm − 2a |
| 16, 17, 18, 19, 20 | 7 × 0.2 cm − 4a |
| 31, 32, 33, 34, 35, 36, 37, 38, 40, 23 | 14 × 0.2 cm − 4a |
| 25, 42, 43, 44, 45, 46, 47, 48, 49, 50 | 14 × 0.2 cm − 5a |
| 51, 52, 53, 54 | Negative Control |
| 55, 56, 57, 58 | Positive Control |

| | | |
|---|---|---|
| 10/5/04 | Day 0 | Weigh pigs. |
| | | Implant according to treatment schedule. |
| | | Treat daily PST pigs. |
| | | Measure and record P2. |
| 11/5/04 | Day 1 | Record feed refusals and feed offered. |
| | | Treat daily PST pigs. |
| 12/5/04 | Day 2 | Record feed refusals and feed offered. |
| | | Treat daily PST pigs. |
| 13/5/04 | Day 3 | Record feed refusals and feed offered. |
| | | Treat daily PST pigs. |
| 14/5/04 | Day 4 | Record feed refusals and feed offered. |
| | | Treat daily PST pigs. |
| | | Weigh pigs. |
| | | Calculate FCR. |
| 15/5/04 | Day 5 | Record feed refusals and feed offered. |
| | | Treat daily PST pigs. |
| 16/5/04 | Day 6 | Record feed refusals and feed offered. |
| | | Treat daily PST pigs. |
| 17/5/04 | Day 7 | Record feed refusals and feed offered. |
| | | Treat daily PST pigs. |
| | | Weigh pigs. |
| | | Calculate FCR. |
| | | Measure and record P2. |
| | | TERMINATE STUDY |

The overall experimental schedule is set out in Table 19 below.

TABLE 19
Experimental Schedule

| | | | | | |
|---|---|---|---|---|---|
| Weekday | Date | Experiment Day | Feed Rec | Weigh | P2 |

PART A

| | | | | | | |
|---|---|---|---|---|---|---|
| Monday | 19 Apr. 2004 | −7 | Select | X | X | |
| Tuesday | 20 Apr. 2004 | −6 | Allocate | X | | |
| Wednesday | 21 Apr. 2004 | −5 | | X | | |
| Thursday | 22 Apr. 2004 | −4 | | X | | |
| Friday | 23 Apr. 2004 | −3 | | X | | |
| Saturday | 24 Apr. 2004 | −2 | | X | | |
| Sunday | 25 Apr. 2004 | −1 | | X | | |
| Monday | 26 Apr. 2004 | 0 | Implant | X | X | X |
| Tuesday | 27 Apr. 2004 | 1 | | X | | |
| Wednesday | 28 Apr. 2004 | 2 | | X | | |
| Thursday | 29 Apr. 2004 | 3 | | X | | |
| Friday | 30 Apr. 2004 | 4 | | X | X | |
| Saturday | 1 May 2004 | 5 | | X | | |
| Sunday | 2 May 2004 | 6 | | X | | |
| Monday | 3 May 2004 | 7 | | X | X | X |
| Tuesday | 4 May 2004 | 8 | | X | | |
| Wednesday | 5 May 2004 | 9 | | X | | |
| Thursday | 6 May 2004 | 10 | | X | X | |
| Friday | 7 May 2004 | 11 | | X | | |
| Saturday | 8 May 2004 | 12 | | X | | |
| Sunday | 9 May 2004 | 13 | | X | | |
| Monday | 10 May 2004 | 14 | | X | X | X |

PART B

| | | | | | |
|---|---|---|---|---|---|
| Monday | 10 May 2004 | 0 | X | X | X |
| Tuesday | 11 May 2004 | 1 | X | | |
| Wednesday | 12 May 2004 | 2 | X | | |
| Thursday | 13 May 2004 | 3 | X | | |
| Friday | 14 May 2004 | 4 | X | X | |
| Saturday | 15 May 2004 | 5 | X | | |
| Sunday | 16 May 2004 | 6 | X | | |
| Monday | 17 May 2004 | 7 | X | X | X |

The results achieved in Part B are illustrated in Table 20 below.

Table 20 provides the surprising result that a superior feed conversion efficiency and equivalent backfat reduction may be achieved utilising half the daily equivalent dosage relative to daily injection.

TABLE 20

| | | | | Daily | | | Improvement | |
|---|---|---|---|---|---|---|---|---|
| | | | Length/ | Equiv | Weight | Feed | over | |
| Implant | | | No. of | Dose | Change kg | Conversion | untreated | P2 (mm) |
| Name | Treatment | | Implants | (mg) | Day 0-7 | Ratio | Control | Day 7 |
| 2a | 20% | PST | 0.2 cm × 14 | 6 | 8.6 | 2.27 | 17.5% | 11.2 |
| | 5% | Salt | | | | | | |
| 4a | 20% | PST | 0.2 cm × 7 | 3 | 9.2 | 2.07 | 24.7% | 12.0 |
| | 10% | Salt | | | | | | |
| 5a | 20% | PST | 0.2 cm × 14 | 6 | 10.1 | 2.21 | 19.6% | 11.2 |
| | 15% | Protein | | | | | | |

| | | | | | | |
|---|---|---|---|---|---|---|
| Daily | — | 6 | 9.7 | 2.16 | 21.5% | 13.5 |
| Injection | | | | | | |
| Control | — | — | 9.1 | 2.75 | — | 17.1 |

It will be understood that the invention disclosed and defined in this specification extends to all alternative combinations of two or more of the individual features mentioned or evident from the text or drawings. All of these different combinations constitute various alternative aspects of the invention.

It will also be understood that the term “comprises” (or its grammatical variants) as used in this specification is equivalent to the term “includes” and should not be taken as excluding the presence of other elements or features.

1. A sustained release delivery apparatus including
a silicone support material formed from a methyl-vinyl siloxane polymer including a fumed silica as a reinforcing filler;
a pharmaceutically active composition carried in or on the silicone support material;
the pharmaceutically active composition including at least one growth and/or reproduction-associated pharmaceutical component; analogue thereof or derivative thereof; and
a carrier therefor.

2. A sustained release apparatus according to claim 1 wherein the apparatus exhibits loading capacities of growth and/or reproduction-associated pharmaceutical active of approximately 10% to 65% by weight, based on the total weight of the pharmaceutically active composition.

3. A sustained release apparatus according to claim 1, wherein the silicone support material has a molded or extruded rod structure.

4. A sustained release apparatus according to claim 3, wherein the silicone support material has a coated rod structure.

5. A sustained release apparatus according to claim 4, wherein the silicone support material has a co-extruded rod structure.

6. A sustained release apparatus according to claim 1, wherein the apparatus provides approximately zero order release of pharmaceutical active.

7. A sustained release apparatus according to claim 1, wherein the pharmaceutical active is selected from one or more of the group consisting of cytokines, hormones, growth factors, live vectors and live cells secreting growth hormones and RNA and DNA coding for growth hormones.

8. A sustained release apparatus according to claim 7, wherein the pharmaceutical active includes recombinant porcine somatotropin (rPST).

9. A sustained release apparatus according to claim 8, wherein the pharmaceutical active further includes at least one pharmaceutically active component selected from the group consisting of acetonemia preparations, anabolic agents, anaesthetics, analgesics, anti-acid agents, anti-arthritic agents, antibodies, anti-convulsivants, anti-fungals, anti-histamines, anti-infectives, anti-inflammatories, anti-microbials, anti-parasitic agents, anti-protozoals, anti-ulcer agents, antiviral pharmaceuticals, behaviour modification drugs, biologicals, blood and blood substitutes, bronchodilators and expectorants, cancer therapy and related pharmaceuticals, cardiovascular pharmaceuticals, central nervous system pharmaceuticals, coccidiostats and coccidiocidals, contraceptives, contrast agents, diabetes therapies, diuretics, fertility pharmaceuticals, hematinics, hemostatics, hormone replacement therapies, hormones and analogs, immunostimulants, minerals, muscle relaxants, natural products, nutraceuticals and nutritionals, obesity therapeutics, ophthalmic pharmaceuticals, osteoporosis drugs, pain therapeutics, peptides and polypeptides, respiratory pharmaceuticals, sedatives and tranquilizers, transplantation products, urinary acidifiers, vaccines and adjuvants and vitamins.

10. A sustained release apparatus according to claim 8, wherein the pharmaceutical active further includes a vaccine component selected from one or more of the group consisting of vaccines against Adenovirus, Anthrax, BCG, Chlamydia, Cholera, Circovirus, Classical swine fever, Coronavirus, Diphtheria-Tetanus, Distemper virus, DTaP, DTP, E. coli, Eimeria (coccidosis), Feline immunodeficiency virus, Feline leukemia virus, Foot and mouth disease, Hemophilus, Hepatitis A, Hepatitis B, Hepatitis B/Hib, Herpes virus, Hib, Influenza, Japanese Encephalitis, Lyme disease, Measles, Measles-Rubella, Meningococcal, MMR, Mumps, Mycoplasma, Para influenza virus, Parvovirus, Pasteurella, Pertussis, Pestivirus, Plague, Pneumococcal, Polio (IPV), Polio (OPV), Pseudorabies, Rabies, Respiratory syncitial virus, Rotavirus, Rubella, Salmonella, Tetanus, Typhoid, Varicella and Yellow Fever.

11. A sustained release apparatus according to claim 1, wherein the pharmaceutical carrier is selected to permit release of the pharmaceutically active component from the composition over an extended period of time and includes a water-soluble substance which is in a solid state in the pharmaceutically active composition at the body temperature of an animal or human being to which it is to be administered.

12. A sustained release apparatus according to claim 11, wherein the pharmaceutical carrier is selected from one or more of the group consisting of synthetic polymers, sugars, amino acids, mineral salts, organic salts and proteins.

13. A sustained release apparatus according to claim 12, wherein the pharmaceutical carrier is a protein or mineral salt, or mixture thereof.

14. A sustained release apparatus according to claim 1 including a plurality of sustained release mini-implants or pellets;
each mini-implant including
a silicone support material formed from a methyl-vinyl siloxane polymer including a fumed silica as a reinforcing filler; and
a pharmaceutically active composition carried in or on the silicone support material;
the pharmaceutically active composition including
at least one growth and/or reproduction-associated pharmaceutical; analogue thereof or derivative thereof; and
a carrier therefor;
each implant being of insufficient size and/or payload individually to provide a predetermined desired threshold blood level of pharmaceutical active for treatment of a selected growth and/or reproduction-associated indication.

15. A sustained release apparatus according to claim 14, wherein the mini-implants provide, in use, less than the daily equivalent injectable dosage.

16. A sustained release apparatus according to claim 15, wherein the mini-implants provide, in use, approximately half the daily equivalent injectable dosage.

17. A sustained release apparatus according to claim 14, including 1 to approximately 20 mini implants.

18. A sustained release apparatus according to claim 17, including approximately 5 to 20 mini-implants.

19. A sustained release apparatus according to claim 17, wherein each mini-implant is of the uncovered or covered rod type, and has an axial length of approximately 1 to 40 mm.

20. A sustained release apparatus according to claim 19, wherein each mini-implant has an axial length of approximately 1 to 5 mm.

21. A sustained release apparatus according to claim 20, wherein each mini-implant has an axial length of approximately 2 mm.

22. A sustained release apparatus according to claim 14, wherein the silicone support material has a co-extruded rod structure.

23. A sustained release apparatus according to claim 14, wherein each mini-implant includes
a pharmaceutical active-containing inner layer; and
a water-impermeable outer layer.

24. A sustained release apparatus according to claim 23, wherein each mini-implant takes the form of a co-extruded rod.

25. A sustained release composition in a unit dosage form including
at least one growth and/or reproduction-associated pharmaceutical component, analogue thereof or derivative thereof; and
a non-silicone pharmaceutical carrier therefor;
wherein said composition includes
approximately 1% to 20% by weight alkali metal chloride;
approximately 0.5% to 5% by weight lubricant; and
approximately 75% to 97.5% by weight growth and/or reproduction-associated pharmaceutical component.

26. A sustained release unit dosage composition according to claim 25, wherein the pharmaceutical active is selected from one or more of the group consisting of cytokines, hormones, growth factors, live vectors and live cells secreting growth hormones and RNA and DNA coding for growth hormones.

27. A sustained release unit dosage composition according to claim 26, wherein the pharmaceutical component is selected from the group consisting of one or more of growth hormones, growth hormone releasing factor, calcitonin, leuteinizing hormone, leuteinizing hormone releasing hormone and insulin.

28. A sustained release unit dosage composition according to claim 27, wherein the growth hormone is a natural or synthetic human, porcine, bovine, canine, feline, piscine or ovine growth hormone.

29. A sustained release unit dosage composition according to claim 25, where the composition is in the form of a mini-tablet implant.

30. A sustained release unit dosage composition according to claim 25, including
approximately 5% to 15% by weight sodium chloride;
approximately 0.5% to 5% by weight magnesium stearate; and
approximately 80% to 94.5% by weight recombinant porcine somatotropin.

31. A sustained release kit including a plurality of sustained release mini-implants or pellets packaged for delivery in a single treatment,
each mini-implant including
a silicone support material formed from a methyl-vinyl siloxane polymer including a fumed silica as a reinforcing filler; and
a pharmaceutically active composition carried in or on the silicone support material;
the pharmaceutically active composition including
at least one growth and/or reproduction-associated pharmaceutical; analogue thereof or derivative thereof; and
a carrier therefor;
each implant being of insufficient size and/or payload individually to provide a predetermined desired threshold blood level of pharmaceutical active for treatment of a selected growth and/or reproduction-associated indication.

32. A sustained release kit according to claim 31, wherein the mini-implants provide, in use, approximately half the daily equivalent injectable dosage.

33. A sustained release kit according to claim 31 including approximately 1 to 20 mini-implants.

34. A sustained release kit according to claim 33, including approximately 5 to 20 mini-implants.

35. A sustained release kit according to claim 34, wherein each mini-implant is of the uncovered or covered rod type, and has an axial length of approximately 1 to 40 mm.

36. A sustained release kit according to claim 35, wherein each mini-implant has an axial length of approximately 1 to 5 mm.

37. A sustained release kit according to claim 36, wherein each mini-implant has an axial length of approximately 2 mm.

38. A sustained release kit according to claim 31, wherein the silicone support material has a co-extruded rod structure.

39. A sustained release kit according to claim 31, wherein the mini-implants are packaged in a biodegradable sheath.

40. A sustained release kit including at least one sustained release mini tablet implant packaged for delivery in a single treatment,
the or each mini tablet implant including
a sustained release composition in a unit dosage form including
approximately 1% to 20% by weight alkali metal chloride;
approximately 0.5% to 5% by weight lubricant; and
approximately 75% to 97.5% by weight growth and/or
reproduction-associated pharmaceutical component;
a non-silicone pharmaceutical carrier therefor;
the or each implant together being of substantially reduced size and/or payload relative to an equivalent immediate release treatment.

41. A sustained release kit according to claim 40, wherein, when a plurality of sustained release mini tablets implants are used, each implant is of insufficient size and/or payload individually to provide a predetermined required threshold blood level of pharmaceutical active for treatment of a selected indication.

42. A sustained release kit according to claim 41, wherein the multiple sustained release mini tablet implants are packaged in a biodegradable sheath.

43. A method for the therapeutic or prophylactic treatment of a condition in an animal (including a human) requiring such treatment, or to improve a physiological characteristic of an animal, which method includes administering to the animal a sustained release composition in a unit dosage form including
approximately 1% to 20% by weight alkali metal chloride;
approximately 0.5% to 5% by weight lubricant; and
approximately 75% to 97.5% by weight growth and/or reproduction-associated pharmaceutical component and
a non-silicone pharmaceutical carrier therefor;

44. A method according to claim 43, wherein the improved nutritional and/or growth-related characteristics are selected from one or more of the group consisting of growth rate, feed conversion ratio, backfat measurement, plasma urea concentrations and plasma glucose levels.

45. A method according to claim 44, wherein the pharmaceutical active is selected from one or more of cytokines, hormones, growth factors, or mixtures thereof, live vectors and live cells secreting growth hormones and RNA and DNA coding for growth hormones.

46. A method according to claim 45, wherein the pharmaceutical active includes recombinant porcine somatotropin (rPST).

47. A method according to claim 43, which method includes administering to the animal a sustained release delivery apparatus including
a silicone support material formed from a methyl-vinyl siloxane polymer including a fumed silica as reinforcing filler;
a pharmaceutically active composition carried in or on the silicone support material;
the pharmaceutically active composition including
at least one growth and/or reproduction-associated pharmaceutical component; analogue thereof or derivative thereof; and
a carrier therefor.

48. A method for the therapeutic or prophylactic treatment of an animal (including a human) to improve a nutritional and/or growth-related characteristic of an animal, which method includes
administering to the animal
at least one sustained release delivery apparatus including
a silicone support material formed from a methyl-vinyl siloxane polymer including a fumed silica as reinforcing filler; and
a pharmaceutical composition carried in or on the support material including
at least one growth-associated pharmaceutical component an analogue thereof or derivative thereof; and
a carrier therefor;
the sustained release delivery apparatus exhibiting generally zero order release; the size and/or number thereof being selected to improve at least one growth-associated physiological characteristic.

49. A method according to claim 48, wherein the improved nutritional and/or growth-related characteristics are selected from one or more of the group consisting of growth rate, feed conversion ratio, backfat measurement, plasma urea concentrations and plasma glucose levels.

50. A method according to claim 49, wherein the pharmaceutical active is selected from one or more of cytokines, hormones, growth factors, or mixtures thereof, live vectors and live cells secreting growth hormones and RNA and DNA coding for growth hormones.

51. A method according to claim 50, wherein the pharmaceutical active includes recombinant porcine somatotropin (rPST).

52. A method according to claim 48, wherein the sustained release delivery apparatus includes a plurality of sustained release mini-implants;
each implant being of insufficient size and/or payload individually to provide a predetermined desired threshold blood level of pharmaceutical active for treatment of a selected growth and/or reproduction-associated indication.

53. A method according to claim 52, wherein the feed conversion ratio is improved over an extended period of time and backfat reduction is maintained for a further extended period of time.

54. A method according to claim 53, wherein, when the animal is a pig, the feed conversion ratio is improved for at least approximately 7 days and backfat reduction is maintained for at least approximately 14 days.

55. A method according to claim 52, wherein the mini-implants provide, in use, less than the daily equivalent injectable dosage.

56. A method according to claim 55, wherein the mini-implants provide, in use, approximately half the daily equivalent injectable dosage.

57. A method according to claim 52, wherein the sustained release apparatus includes 1 to approximately 20 implants.

58. A method according to claim 57, wherein the sustained release apparatus includes approximately 5 to 20 mini-implants.

59. A method according to claim 58, wherein each mini-implant is of the uncovered or covered rod type, and has an axial length of approximately 1 to 40 mm.

60. A method according to claim 59, wherein each mini-implant has an axial length of approximately 1 to 5 mm.

61. A method according to claim 59, wherein, when the animal is a pig, 7 to 20 mini-implants each having an axial length of approximately 2 mm, are administered.

62. A method according to claim 52, wherein the silicone support material has a co-extruded rod structure.

63. A method according to claim 52, wherein the mini implants or pellets are administered via any one or more of the routes selected from the group consisting of subcutaneous, intraperitoneal intramuscular injection, intranasal insertion or indwelling, intrarectal insertion or indwelling.

64. A method according to claim 52, wherein the animal to be treated is selected from the group consisting of sheep, cattle, goats, horses, camels, pigs, dogs, cats, ferrets, rabbits, marsupials, buffalos, yacks, primates, humans, birds including chickens, geese and turkeys, rodents including rats and mice, fish, reptiles and the like.

65. A method according to claim 64, wherein the animal to be treated is selected from cattle, sheep, pigs, dogs and humans.
